(19) United States
(12) Patent Application Publication
(10) Pub. No.: US 2026/0083806 A1
(43) Pub. Date: Mar. 26, 2026

(54) Heart boosting formulation and related methods of manufacture

(71) Applicant: Kedar Prasad, San Rafael, CA (US)

(72) Inventor: Kedar Prasad, San Rafael, CA (US)

(21) Appl. No.: 18/831,814

(22) Filed: Sep. 16, 2025

Related U.S. Application Data
(60) Provisional application No. 63/732,775, filed on Sep. 23, 2024.

(51) Int. Cl. A61K 36/54; A61K 31/01; A61K 31/198; A61K 31/202; A61K 31/205; A61K 31/593; A61K 36/21; A61K 36/53; A61P 9/00

(52) U.S. Cl. CPC A61K 36/54; A61K 31/01; A61K 31/198; A61K 31/202; A61K 31/205; A61K 31/593; A61K 36/21; A61K 36/53; A61P 9/00

(57) ABSTRACT

A heart boosting formulation comprises: L-carnitine, alpha-linolenic acid, citrulline, vitamin D3, lycopene, holy basil, cinnamon and beetroot.

### RELATED APPLICATIONS

This application claims priority to the Provisional Application No. 63/732,775 filed on Sep. 23, 2024 entitled “Heart Boosting Formulation and Related Methods of Manufacture’.

### BACKGROUND OF THE INVENTION

Cardiovascular disease, which includes coronary artery disease, heart disease, and stroke is the leading cause of death in men and women in the USA. The CDC (Center for Disease Control and Prevention, 2023) estimated that one person dies every 33 seconds in the USA. In 2021, 695,000 people died of heart disease. Between 2018-2019, the national cost of treatment of heart disease each year was 239.9 billion dollars. High blood pressure, high blood level of cholesterol, and smoking are the major risk factors for developing cardiovascular disease. Other risk factors include diabetes, obesity, lack of physical activity, unhealthy diet, and excessive alcohol consumption. Exposure to these risk factors increase oxidative stress and chronic inflammation in the body which play a central role in the development and progression of cardiovascular disease. Therefore, it is proposed that improving diet, lifestyle (stopping smoking, increasing physical activity), reducing high blood pressure and high blood cholesterol level, and improving management of diabetes together with decreasing the level of oxidative stress and chronic inflammation by adopting Microdaily EMF and heart boost may help in prevention and improved management of heart disease.

### SUMMARY OF THE INVENTION

In one embodiment, the present invention relates to a formulation comprising L-carnitine, alpha-linolenic acid, citrulline, vitamin D3, lycopene, holy basil, cinnamon and beetroot. In another embodiment, the formulation functions as a heart booster or heart boosting formula.

In yet another further embodiment, the amount of said L-carnitine in the formulation is from about 50 mg to about 500 mg by weight of the formulation. In still another embodiment, the amount of the alpha-linolenic acid in the formulation is from about 50 mg to about 800 mg by weight of the formulation. In a still yet another embodiment, the amount of the citrulline in the formulation is from about 50 mg to about 500 mg by weight of the formulation. In a further embodiment, the amount of said vitamin D3 in the formulation is from about 500 IU to about 2500 IU by weight of the formulation. In still a further embodiment, the amount of the lycopene in the formulation is from about 10 mg to about 40 mg by weight of the formulation. In yet a further embodiment, the amount of the holy basil in the formulation is from about 50 mg to about 500 mg by weight of the formulation. In another further embodiment, the amount of the cinnamon in the formulation is from about 50 mg to about 400 mg by weight of the formulation. In yet another further embodiment, the amount of the beetroot in the formulation is from about 25 mg to about 250 mg by weight of the formulation.

In another embodiment, a therapeutically effective amount of the formulation is intended to be administered at least once a day. In a further embodiment, a therapeutically effective amount of the formulation is intended to be administered twice a day. In another embodiment, the formulation functions as a heart booster.

In another embodiment, the present invention relates to a formulation comprising active agents, said active agents consisting of L-carnitine, alpha-linolenic acid, citrulline, vitamin D3, lycopene, holy basil, cinnamon and beetroot. In a further embodiment, the formulation further comprises a carrier, preservatives and additives. In still a further embodiment, the carrier is in a form selected from the group consisting of: liquid, pill, hard shell capsule, soft shell capsule, gel, tablet, ointment, solution and combinations thereof.

In a further embodiment, the active agents comprise L-carnitine from about 50 mg to about 500 mg by weight of said formulation, alpha-linolenic acid from about 50 mg to about 800 mg by weight of said formulation, citrulline from about 50 mg to about 500 mg by weight of said formulation, vitamin D3 from about 500 IU to about 2500 IU by weight of said formulation, lycopene from about 10 mg to about 40 mg by weight of said formulation, holy basil from about 50 mg to about 500 mg by weight of said formulation, cinnamon from about 50 mg to about 400 mg by weight of said formulation, and beetroot from about 25 mg to about 250 mg by weight of said formulation.

In another further embodiment, the present invention provides for a method of manufacturing a formulation, said method comprises: admixing L-carnitine, alpha-linolenic acid, citrulline, vitamin D3, lycopene, holy basil, cinnamon and beetroot. In yet another further embodiment, the method further comprises: providing said formulation with a carrier. In still another embodiment, the carrier is in a form selected from the group consisting of: liquid, pill, hard shell capsule, soft shell capsule, gel, tablet, ointment, solution and combinations thereof.

In another embodiment, the method uses L-carnitine from about 50 mg to about 500 mg by weight of said formulation, alpha-linolenic acid from about 50 mg to about 800 mg by weight of said formulation, citrulline from about 50 mg to about 500 mg by weight of said formulation, vitamin D3 from about 500 IU to about 2500 IU by weight of said formulation, lycopene from about 10 mg to about 40 mg by weight of said formulation, holy basil from about 50 mg to about 500 mg by weight of said formulation, cinnamon from about 50 mg to about 400 mg by weight of said formulation, and beetroot from about 25 mg to about 250 mg by weight of said formulation.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

### Relationship Between the Components:

A comprehensive mixture of micronutrients contains L-carnitine, alpha-linolenic acid, citrulline, vitamin D3, lycopene, tulsi or tulasi (aka Holy Basil), cinnamon, and beetroot.

Effects of L-carnitine on heart function: Although β-blockers and inhibitors of angiotensin-converting-enzymes ACE) are commonly prescribed to control cardiovascular disease (CVD) risk, they produce adverse side-effects on the liver and hematopoietic system Recent studies suggest that L-carnitine helps to transport of long-chain fatty acids into the mitochondrial matrix, causing reduced oxidative stress, inflammation and preventing necrosis of cardiac myocytes. In addition, l-carnitine has an important role in regulating calcium influx and endothelial integrity. Therefore, supplementation with L-carnitine may provide protection against ventricular dysfunction, ischemia-reperfusion injury, cardiac arrhythmia, and myocardial injury that is often associated with CVD. Furthermore, supplementation with L carnitine may reduce hypertension, hyperlipidemia, diabetic ketoacidosis, hyperglycemia, insulin-dependent diabetes mellitus, insulin resistance, and obesity that are known to increase the risk of heart disease. Supplementation with L-carnitine improved clinical symptoms and cardiac function in chronic heart failure.

Effects of alpha-linolenic acid on heart function: Epidemiologic and dietary trials in humans suggest that alpha-Linolenic acid (ALA) is a major cardio-protective agent. It helps to maintain normal heart rhythm and may prevent ventricular fibrillation. Higher blood level of alpha-ALA is associated with a reduced risk of cardiovascular disease (CVD) and coronary artery disease. A review has suggested that alpha-ALA plays a beneficial role in primary and secondary prevention of CVD.

Effects of citrulline on heart function: L-citrulline is a precursor of L-arginine which produces nitric oxide (NO) that dilates the blood vessels. L-arginine has been shown to improve cardiovascular function such as hypertension, atherosclerosis, diabetic vascular disease, and ischemia-reperfusion injury. However, the effectiveness of L-arginine supplementation does not last long because of induction of increased activity of the enzyme arginase which degrades L-arginine. In contrast, L-citrulline inhibits arginase activity raising the plasma levels of arginine. Daily supplementation with a dose of 6 g/day for 7 days improved brachial artery endothelium-dependent vasodilation, upper and lower limb microvascular function, and physical capacity in patients with heart failure with a preserved ejection fraction. Citrulline supplementation lowers systolic and diastolic blood pressure. Supplementation with s daily dose of 3 g/day for 4 months improved the left ventricular ejection fraction, endothelial function, and functional class in patients with systolic heart failure.

Effects of vitamin D3 on heart function: Vitamin D3 can repair damage to the heart and blood vessels caused by high blood pressure, A large clinical study suggests that supplementation with vitamin D3 might decrease the incidence of major cardiovascular events. Another clinical study showed that supplementation with vitamin D3 may benefit in atrial fibrillation prevention in elderly population. Vitamin D3 deficiency is associated with morbidity and mortality in heart failure patients. Repleting vitamin D3 in your body improves quality of life in heart failure patients and may help to normalize the levels of parathyroid hormone, b-type natriuretic peptide, and highly sensitive C-reactive protein.

Effects of lycopene on heart function: Increased oxidative stress can lead to cardiac hypertrophy caused by pressure overload. Lycopene can inhibit the development of cardiac hypertrophy by reducing oxidative stress and reduced systolic blood pressure. This carotenoid is 10 times more potent than vitamin E and 2 times more effective than beta-carotene. There are two isoforms of lycopene all-trans and cis. Fresh tomato contains 90% all-trans lycopene, while catchup has mostly cis-lycopene. The absorption of cis-lycopene is much higher than all-trans lycopene. Lycopene reduces oxidative stress and chronic inflammation.

Effects of Tulsi (Holy Basil) on heart function: Analysis of 24 human short-term studies suggest that supplementation with Tulsi extract reduces lipid contents, suppresses ischemia and stroke, lowers hypertension, normalizes blood sugar, improves memory, and reduces psychological stresses such as anxiety and depression.

Effects of cinnamon on heart function: Cinnamon is important for maintaining a healthy heart. This spice reduces blood cholesterol and triglycerides levels. In addition, it exhibits antioxidant and anti-inflammation activities, protects endothelial cells, improves immune response, suppresses smooth cells growth and metabolism, platelet activity, and thrombosis, and inhibits angiogenesis. Supplementation with cinnamon reduces the risk of developing cardiovascular disease in patients with diabetes by enhancing the sensitivity of insulin. In patients with stage I hypertension, daily supplementation with a dose of 1500 mg/day for 90 days decreased systolic blood pressure (BSP) and improved lipid profiles.

Effects of Beetroot on heart function: In a clinical study on participants aged 35-65 years, daily supplementation with beetroot powder at a dose of 500 mg/day for 4 weeks showed improvement in cardiovascular function. A combination with vitamin C produced a synergistic effect on the improvement of cardiovascular function. Beetroot is very rich in nitrate which increases the production of NO (nitric oxide) that acts as a vasodilator. On the other hand, vitamin C in combination with beetroot enhances the ability of cells to produce more NO.

In one of the embodiments of the present invention, Table 1, set forth below, represents a formulation for functioning as a heart boost:

TABLE 1

| Active Agents: | % By Weight of Formulation |
|---|---|
| L-carnitine | from about 50 mg to about 500 mg |
| alpha-linolenic acid | from about 50 mg to about 800 mg |
| citrulline | from about 50 mg to about 500 mg |
| vitamin D3 | from about 500 IU to about 2500 IU |
| lycopene | from about 10 mg to about 40 mg |
| holy basil | from about 50 mg to about 500 mg |
| cinnamon | from about 50 mg to about 400 mg |
| beetroot | from about 25 mg to about 250 mg |

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

Numerous modifications and variations of the present invention are possible in light of the above teachings. It is therefore to be understood that within the scope of the attendant claims attached hereto, this invention may be practiced otherwise than as specifically disclosed herein.

What is claim is:

1. A formulation comprising L-carnitine, alpha-linolenic acid, citrulline, vitamin D3, lycopene, holy basil, cinnamon and beetroot.

2. The formulation of claim 1 wherein the amount of said L-carnitine is from about 50 mg to about 500 mg by weight of said formulation.

3. The formulation of claim 1 wherein the amount of said alpha-linolenic acid is from about 50 mg to about 800 mg by weight of said formulation.

4. The formulation of claim 1 wherein the amount of said citrulline is from about 50 mg to about 500 mg by weight of said formulation.

5. The formulation of claim 1 wherein the amount of said vitamin D3 is from about 500 IU to about 2500 IU by weight of said formulation.

6. The formulation of claim 1 wherein the amount of said lycopene is from about 10 mg to about 40 mg by weight of said formulation.

7. The formulation of claim 1 wherein the amount of said holy basil is from about 50 mg to about 500 mg by weight of said formulation.

8. The formulation of claim 1 wherein the amount of said cinnamon is from about 50 mg to about 400 mg by weight of said formulation.

9. The formulation of claim 1 wherein the amount of said beetroot is from about 25 mg to about 250 mg by weight of said formulation.

10. The formulation of claim 1 wherein a therapeutically effective amount of said formulation is intended to be administered at least once a day.

11. The formulation of claim 1 wherein a therapeutically effective amount of said formulation is intended to be administered should be taken twice a day.

12. The formulation of claim 1 wherein said formulation functions as a heart booster.

13. A formulation comprising active agents, said active agents consisting of L-carnitine, alpha-linolenic acid, citrulline, vitamin D3, lycopene, holy basil, cinnamon and beetroot.

14. The formulation of claim 13 further comprises a carrier, preservatives and additives.

15. The formulation of claim 14 wherein said carrier is in a form selected from the group consisting of: liquid, pill, hard shell capsule, soft shell capsule, gel, tablet, ointment, solution and combinations thereof.

16. The formulation of claim 13 wherein the amount of said L-carnitine is from about 50 mg to about 500 mg by weight of said formulation, the amount of said alpha-linolenic acid is from about 50 mg to about 800 mg by weight of said formulation, the amount of said citrulline is from about 50 mg to about 500 mg by weight of said formulation, the amount of said vitamin D3 is from about 500 IU to about 2500 IU by weight of said formulation, the amount of said lycopene is from about 10 mg to about 40 mg by weight of said formulation, the amount of said holy basil is from about 50 mg to about 500 mg by weight of said formulation, the amount of said cinnamon is from about 50 mg to about 400 mg by weight of said formulation, and the amount of said beetroot is from about 25 mg to about 250 mg by weight of said formulation.

17. A method of manufacturing a formulation, said method comprises: admixing L-carnitine, alpha-linolenic acid, citrulline, vitamin D3, lycopene, holy basil, cinnamon and beetroot.

18. The method of claim 17 further comprising: providing said formulation with a carrier.

19. The method of claim 17 wherein said carrier is in a form selected from the group consisting of: liquid, pill, hard shell capsule, soft shell capsule, gel, tablet, ointment, solution and combinations thereof.

20. The method of claim 17 wherein the amount of said L-carnitine is from about 50 mg to about 500 mg by weight of said formulation, the amount of said alpha-linolenic acid is from about 50 mg to about 800 mg by weight of said formulation, the amount of said citrulline is from about 50 mg to about 500 mg by weight of said formulation, the amount of said vitamin D3 is from about 500 IU to about 2500 IU by weight of said formulation, the amount of said lycopene is from about 10 mg to about 40 mg by weight of said formulation, the amount of said holy basil is from about 50 mg to about 500 mg by weight of said formulation, the amount of said cinnamon is from about 50 mg to about 400 mg by weight of said formulation, and the amount of said beetroot is from about 25 mg to about 250 mg by weight of said formulation.
